(19) United States
(12) Patent Application Publication
(10) Pub. No.: US 2004/0121017 A1
(43) Pub. Date: Jun. 24, 2004

(54) Polymer gel composition, and optical device, resin composition and optical film using the polymer gel composition

(75) Inventors: Rie Ishii, Minamiashigara-shi (JP); Akinori Komura, Minamiashigara-shi (JP); Akira Imai, Minamiashigara-shi (JP); Masahiro Moriyama, Minamiashigara-shi (JP); Ryojiro Akashi, Minamiashigara-shi (JP)

Correspondence Address: OLIFF & BERRIDGE, PLC, P.O. BOX 19928, ALEXANDRIA, VA, 22320, US

(73) Assignee: Fuji Xerox Co., Ltd., Tokyo (JP)

(21) Appl. No.: 10/623,539

(22) Filed: Jul. 22, 2003

(30) Foreign Application Priority Data
Dec. 13, 2002 (JP) 2002-362002

(51) Int. Cl. A61K 9/14

(52) U.S. Cl. 424/487

(57) ABSTRACT

The present invention relates to a polymer gel composition including: a swelling liquid; and a polymer gel having characteristics of absorbing and releasing the swelling liquid due to a change in temperature to thereby change a volume thereof reversibly, and including a crosslinked polymer having at least a hydrogen bonding group, wherein the crosslinked polymer included in the polymer gel is a copolymer of at least two monomer components including a (meth)acrylamide or a mono-substituted derivative thereof as a monomer component (A) and a monomer different from the monomer component (A) as a monomer component (B). The invention also relates to an optical device, a resin composition and an optical film, all using the polymer gel composition.

### CROSS-REFERENCE TO RELATED APPLICATION

This application claims priority under 35USC 119 from Japanese Patent Application No. 2002-362002, the disclosure of which is incorporated by reference herein.

### BACKGROUND OF THE INVENTION

1. Field of the Invention

The present invention relates to a polymer gel composition including a polymer gel which has the characteristic of absorbing and releasing a swelling liquid due to a change in temperature to thereby change a volume thereof reversibly; and to an optical device, a resin composition and an optical film, all using the polymer gel composition.

2. Description of the Related Art

Hitherto, there has been known a polymer gel (hereinafter referred to as a stimuli-responsive polymer gel) causing a volume change (swelling or shrinking) thereof under influences of a pH value, an ion concentration, adsorption/desorption of a chemical material or addition of a solvent; or alternatively, by application of heat, light, current, an electric field or the like, and the polymer gel is expected to be of service in application as functional materials. Such functional materials are described, for example, in “Kinousei Koubunshi Gel” (Functional Polymer Gel), from CMC Publishing Co., Ltd. as a general introduction thereof. As applications of the stimuli-responsive polymer gel, there have been investigated: a drug carrier in a drug delivery system or the like, a medical material, an additive to an ink, a functional film, an artificial muscle, a display device, a recording device, an actuator, a pump and others. Generally, by stimulating a stimuli-responsive polymer gel present in a liquid such as water or an electrolyte solution, the polymer gel causes a phase transition or the like thereof to absorb a liquid into inside of the gel or discharge it therefrom, thereby enabling a volume, a size and a shape thereof to be changed.

The inventors have thus far proposed a polymer gel composition including a stimuli-responsive polymer gel and a swelling liquid, together with a production method therefor and further proposed the usage of the polymer gel composition in optical device such as a display device, a recording device, a light-modulation device, a sensor and others (Japanese Patent Application Laid-Open (JP-A) Nos. 11-236559 and 11-228850). Furthermore, the inventors have paid attention to a polymer gel including a polymer gel having a hydrogen bonding group, which changes a degree of the hydrogen bonding and a degree of solvation with a change in temperature, thereby assumes a swelling state at a high temperature, while assuming a shrinking state at a low temperature, and proposed the polymer gel as materials for use in a light-modulation device such as glass and a film that modulates a light depending on an atmospheric temperature, and in thermo-responsive display device.

The inventors have proposed, as in the proposals described above, polymer gels having excellent characteristics, for which various applications have been expected. Depending on an application, durability is required in some cases so as to sustain a light-modulation function over a longer term of usage than in the case of conventional material. Especially, in the case of a polymer gel including a polymer gel having a hydrogen bonding group, which changes a degree of hydrogen bonding and a degree of solvation with a change in temperature and thereby assumes a swelling state at a high temperature, while assuming a shrinking state at a low temperature, it is supposed that the polymer gel has a chance that it is left for a long term at a high temperature, thus further improvement has been desired so that a volume change amount (a volume of a polymer gel in a heated state/a volume thereof in a cooled state) is not reduced; that is so that a light-modulation performance is not lowered (no degradation in characteristiscs).

### SUMMARY OF THE INVENTION

One object of the present invention is to solve the problems in the prior art described above and to thereby achieve the following object. That is, it is an object of the invention to provide a polymer gel composition sustaining a light-modulation performance over a long term without reduction and excellent in durability even at a high temperature and to provide an optical device, a resin composition and an optical film, using the polymer gel composition.

The above object can be achieved according to the following means. That is:

The first aspect of the invention is to provide a polymer gel composition (S) comprising:

a swelling liquid; and

a polymer gel which has the characteristic of absorbing and releasing the swelling liquid due to a change in temperature so as to reversibly change a volume of the polymer gel, and includes a crosslinked polymer having at least a hydrogen bonding group,

wherein the crosslinked polymer included in the polymer gel is a copolymer of at least two monomer components including;

(meth)acrylamide or a mono-substituted derivative thereof as a monomer component (A); and

a monomer different from the monomer component (A) as a monomer component (B).

The second aspect of the invention is to provide a polymer gel composition (S), wherein the monomer component (A) is (meth)acrylamide, and the monomer component (B) is selected from the group consisting of a mono-substituted (meth)acrylamide, a di-substituted (meth)acrylamide, a (meth)acrylate derivative and a vinyl type monomer.

The third aspect of the invention is to provide a polymer gel composition (S), wherein the monomer component (A) is (meth)acrylamide, and the monomer component (B) is selected from the group consisting of a mono-substituted (meth)acrylamide, a di-substituted (meth)acrylamide, and a (meth)acrylate derivative.

The fourth aspect of the invention is to provide a polymer gel composition (S), wherein the monomer component (A) is a mono-substituted derivative of (meth)acrylamide, and the monomer component (B) is selected from the group consisting of (meth)acrylamide, a di-substituted (meth)acrylamide, a (meth)acrylate derivative, and a vinyl type monomer.

The fifth aspect of the invention is to provide a polymer gel composition (S), wherein the monomer component (A) is a mono-substituted derivative of (meth)acrylamide, and the monomer component (B) is selected from a group consisting of (meth)acrylamide, a di-substituted (meth)acrylamide, and a (meth)acrylate derivative.

The sixth aspect of the invention is to provide a polymer gel composition (S), wherein a pH value of the swelling liquid is from 2 to 7.

The seventh aspect of the invention is to provide a polymer gel composition (S), wherein the swelling liquid includes at least a water-soluble organic compound.

The eighth aspect of the invention is to provide a polymer gel composition (S), wherein the swelling liquid includes at least a water-soluble organic compound, and the water-soluble organic compound is an alcohol.

The ninth aspect of the invention is to provide a polymer gel composition (S), wherein the swelling liquid includes at least an acidic compound.

The tenth aspect of the invention is to provide a polymer gel composition (S), wherein the swelling liquid includes at least an acidic compound, and the acidic compound is a polymer having a carboxyl group.

The eleventh aspect of the invention is to provide a polymer gel composition (S), wherein the polymer gel is an IPN material including at least polymers which form hydrogen bonds with each other.

The twelfth aspect of the invention is to provide a polymer gel composition (S), wherein the polymer gel is an IPN material including at least polymers which form hydrogen bonds with each other, and the IPN material comprises the crosslinked polymer having at least a hydrogen bonding group, and a crosslinked polymer having at least a carboxylic group or a partially neutralized crosslinked polymer of the crosslinked polymer having at least a carboxylic group.

The thirteenth aspect of the invention is to provide a polymer gel composition (S), wherein the swelling liquid includes at least an alcohol, and the polymer gel is an IPN material comprising the crosslinked polymer having at least a hydrogen bonding group, and a crosslinked polymer having at least a carboxylic group or a partially neutralized crosslinked polymer of the crosslinked polymer having at least a carboxylic group.

The fourteenth aspect of the invention is to provide a polymer gel composition (S), wherein the swelling liquid includes a polymer having at least a carboxyl group, and the polymer gel is an IPN material comprising the crosslinked polymer having at least a hydrogen bonding group, and a crosslinked polymer having at least a carboxylic group or a partially neutralized crosslinked polymer of the crosslinked polymer having at least a carboxylic group.

The fifteenth aspect of the invention is to provide a polymer gel composition (S), wherein the polymer gel contains a light-modulation material therein.

The sixteenth aspect of the invention is to provide an optical device comprising:

a polymer gel composition comprising

a swelling liquid, and

a polymer gel which has the characteristic of absorbing and releasing the swelling liquid due to a change in temperature so as to reversibly change a volume of the polymer gel, and includes a crosslinked polymer having at least a hydrogen bonding group; and

a substrate sandwiching the polymer gel composition,

wherein the crosslinked polymer included in the polymer gel is a copolymer of at least two monomer components including

(meth)acrylamide or a mono-substituted derivative thereof as a monomer component (A); and

a monomer different from the monomer component (A) as a monomer component (B).

The seventeenth aspect of the invention is to provide a resin composition comprising:

a polymer gel composition comprising

a swelling liquid, and

a polymer gel which has the characteristic of absorbing and releasing the swelling liquid due to a change in temperature so as to reversibly change a volume of the polymer gel, and includes a crosslinked polymer having at least a hydrogen bonding group; and

a resin in which the polymer gel composition is dispersed,

wherein the crosslinked polymer included in the polymer gel is a copolymer of at least two monomer components including (meth)acrylamide or a mono-substituted derivative thereof as a monomer component (A); and

a monomer different from the monomer component (A) as a monomer component (B).

The eighteenth aspect of the invention is to provide an optical film comprising:

a polymer gel composition comprising

a swelling liquid, and

a polymer gel which has the characteristic of absorbing and releasing the swelling liquid due to a change in temperature so as to reversibly change a volume of the polymer gel, and includes a crosslinked polymer having at least a hydrogen bonding group; and

a film on which the polymer gel composition is provided, wherein the crosslinked polymer included in the polymer gel is a copolymer of at least two monomer components including (meth)acrylamide or a mono-substituted derivative thereof as a monomer component (A); and

a monomer different from the monomer component (A) as a monomer component (B).

### BRIEF DESCRIPTION OF THE DRAWINGS

FIG. 1 is a schematic structural view showing an example of a resin composition of the present invention.

FIG. 2 is a schematic structural view showing an example of an optical film of the invention.

FIG. 3 is a schematic structural view showing an example of an optical device of the invention.

### DETAILED DESCRIPTION OF THE INVENTION

Detailed description will be given of the present invention.

A polymer gel composition of the invention includes: a swelling liquid; and a crosslinked polymer compound which has at least a hydrogen bonding group and has the characteristic of absorbing and releasing the swelling liquid due to a change in temperature so as to reversibly change a volume thereof, wherein the crosslinked polymer included in the polymer gel is a copolymer (hereinafter also referred to as a “(meth)acrylamide type polymer”) of at least two monomer components including a (meth)acrylamide or a mono-substituted derivative thereof as a monomer component (A) and a monomer different from the monomer component (A) as a monomer component (B).

A driving mechanism in the change in a volume of a polymer gel including a crosslinked polymer having at least a hydrogen bonding group has been known to be association/dessociation of a hydrogen bond between different kinds of polymer chains (see T. Tanaka, E. Kokufuta et al. Nature, Vol. 349, 400 (1991)). The inventors have found that reduction in volume change amount of a polymer gel composition by heating over a long term is caused by hydrolysis of an amide group of a poly(meth)acrylamide chain, which is a structural constituent of a polymer gel. That is, the inventors inferred that a degradation in characteristics is caused by a reduction in a hydrogen bonding force brought about by hydrolysis.

Therefore, by allowing a polymer gel to include the crosslinked polymer obtained by copolymerizing a (meth)acrylamide or a mono-substituted derivative thereof and a monomer different from the former monomer in order to suppress a degradation in characteristics caused at a high temperature (for example, by heating), self-association between (meth)acrylamides through a hydrogen bond is prevented from occurring and a hydrogen bonding force between different kinds of polymer chains, which is a driving force for a volume change, is strengthened (see T. Okano et al., Macromolecules, Vol. 27, 947(1994)), or a hydrophobic interaction between copolymerization monomers strengthens an interaction between different kinds of polymer chains (see N. Ogata, T. Okano et al., J. Controlled Release, Vol. 16, 215(1991)). Hence, it is supposed that in the invention, a hydrogen boding force is stably sustained even if hydrolysis takes place, thereby degradation in characteristics at a high temperature is minimized.

Furthermore, as is detailed later, it is possible to more effectively prevent degradation in characteristics at a high temperature in a polymer gel composition of the invention by adjusting a pH value of a swelling liquid to fall within the range of 2 to 7, or adding a water-soluble organic compound (an alcohol is especially preferable) into the swelling liquid. This effect is supposed to be caused by a control of a composition of a swelling liquid, which is a reactive solvent, within a condition in which hydrolysis of an amide group included in a polymer gel is hard to occur.

(Polymer Gel)

A polymer gel has a volume phase transition characteristic caused due to a change in degree of hydrogen bonding and degree of solvation with a change in temperature. Generally, a polymer gel absorbs a swelling liquid at a high temperature to assume a swelling state, and releases the swelling liquid at a low temperature to assume a shrinking state. However, a polymer gel having an inverse characteristic is also included.

Such polymer gels include a (meth)acrylamide type polymer (a crosslinked polymer having a hydrogen bonding group), specific examples thereof include an interpenetrating polymer network material (an IPN material) of polymers that form hydrogen bond to each other; and a block copolymer gel comprising at least polymers that form hydrogen bond to each other. Among them, preferable is an interpenetrating polymer network material (an IPN material). As IPN materials, there is preferably exemplified: a gel constituted of a crosslinked polymer of at least two components obtained from the crosslinked (meth)acrylamide type polymer and a crosslinked polymer having at least a carboxyl group. Furthermore, carboxylic groups in a molecule of a polymer may be partially neutralized (a partially neutralized polymer).

A (meth)acrylamide type polymer is a copolymer of at least two monomer components including a (meth)acrylamide or a mono-substituted derivative thereof (hereinafter also referred to as a mono-substituted (meth)acrylamide) as a monomer component (A) and a monomer different from the monomer component (A) as a monomer (B). Specific examples thereof include a copolymer of a (meth)acrylamide (a monomer component (A)) and a monomer (a monomer component (B)) different therefrom; a copolymer of a mono-substituted (meth)acrylamide (a monomer component (A)) and a monomer (a monomer component (B)) different therefrom; a copolymer of a (meth)acrylamide, a mono-substituted (meth)acrylamide (monomer components (A)) and a monomer (a monomer component (B)) different therefrom; and others.

As mono-substituted derivatives of a (meth)acrylamide as a monomer component (A)), there are exemplified: N-methyl(meth)acrylamide, N-ethyl(meth)acrylamide, N-propyl(meth)acrylamide, N-butyl(meth)acrylamide and the like.

On the other hand, as monomer components (B), in the case where a monomer component (A) is a (meth)acrylamide, a monosubstituted (meth)acrylamide, a di-substituted (meth)acrylamide, a (meth)acrylate derivative and a vinyl type monomer are cited as examples, among them, a mono-substituted (meth)acrylamide, a di-substituted (meth)acrylamide and a (meth)acrylate derivative are preferable. As monomer components (B), in the case where a monomer component (A) is a mono-substituted derivative of (meth)acrylamide, a (meth)acrylamide, a di-substituted (meth)acrylamide, a (meth)acrylate derivative and a vinyl type monomer are cited as examples, among them, a (meth)acrylamide, a di-substituted (meth)acrylamide and a (meth)acrylate derivative are preferable. And as monomer components (B), in the case where a monomer component (A) is a (meth)acrylamide and a mono-substituted derivative thereof, a di-substituted (meth)acrylamide, a (meth)acrylate derivative and a vinyl type monomer are cited as examples, among them a di-substituted (meth)acrylamide and a (meth)acrylate derivative are preferable.

As monomer components (B), specific examples thereof include mono-substituted derivatives of a (meth)acrylamide such as an N-methyl(meth)acrylamide, an N-ethyl(meth)acrylamide, an N-propyl(meth)acrylamide, an N-butyl(meth)acrylamide and the like; di-substituted derivatives of a (meth)acrylamide such as, an N,N-dimethyl(meth) acrylamide, an N,N-diethyl(meth)acrylamide, an N,N-dipropyl(meth)acrylamide, an N,N-dibutyl(meth)acrylamide and the like; acrylate derivatives such as a methyl(meth)acrylate, an ethyl(meth)acrylate, a propyl(meth)acrylate, a butyl(meth)acrylate, a hydroxymethyl(meth)acrylate, hydroxyethyl(meth)acrylate, an N,N-dimethylaminoethyl(meth) acrylate, an N,N-diethylaminoethyl(meth)acrylate, a glyceryl(meth)acrylate and the like; and vinyl type monomers such as vinylpyrrolidone, vinyl chloride, vinylidene chloride, styrene, a styrene derivative, isoprene and the like. The polymers obtained from monomers described above can be crosslinked by an optionally copolymerized crosslinker, a chemical reaction, and radiation such as electron beam.

Plural kinds of monomers (A) and plural kinds of monomer components (B) can be used. In this specification, the term “(meth)acryl” includes both meanings of “acryl” and “methacryl.

In a (meth)acrylamide type polymer, a copolymerization molar ratio is preferably in the range of 100/1 to 1/2 in monomer component (A)/monomer component (B). For example a ratio of (meth)acrylamide/a monomer different therefrom, a mono-substituted (meth)acrylamide/a monomer different therefrom, or a (meth)acrylamide/a mono-substituted (meth)acrylamide is preferably within the above range. If the molar ratio is larger than 100/1, an improvement of heat resistance is not obtained in some cases, while if the molar ratio is smaller than 1/2, a desired volume change amount is not obtained in some cases.

On the other hand, specific examples of a crosslinked polymer having at least a carboxyl group include crosslinked polymers obtained by crosslinking polymers such as a poly(meth)acrylic acid, poly-L-glutamic acid, polymaleic acid, polyfumaric acid and the like. Such a polymer having a carboxylic group may also be a copolymer including a different kind of a monomer. A carboxyl groups in a molecule of a polymer described above may be neutralized with a base or the like. A carboxyl group in a polymer described above can be neutralized with, for example, sodium hydroxide, potassium hydroxide, ammonia, amine and the like. A preferable degree of neutralization is from 0 to 20% by mol. The polymers can be crosslinked by an optionally copolymerized crosslinker, a chemical reaction, radiation such as an electron beam.

As interpenetrating polymer network materials (IPN materials) of polymers which form hydrogen bond to each other, specific examples thereof include an IPN material between a crosslinked copolymer comprising at least a (meth)acrylamide and an N,N-dimethyl(meth)acrylamide, and a crosslinked poly(meth)acrylic acid; a partially neutralized material thereof (acrylic acid units in a molecule are partially neutralized into salts); an IPN material between a crosslinked copolymer comprising at least a (meth)acrylamide and a methyl (meth)acrylate, and a crosslinked poly(meth)acrylic acid; and a partially neutralized material thereof (acrylic acid units in a molecule are partially neutralized into salts); an IPN material between a crosslinked copolymer comprising at least a (meth)acrylamide and an ethyl (meth)acrylate, and a crosslinked poly(meth)acrylic acid; and a partially neutralized material thereof (acrylic acid units in a molecule are partially neutralized into salts); an IPN material between a crosslinked copolymer comprising at least a (meth)acrylamide and an ethyl (meth)acrylamide and a crosslinked poly(meth)acrylic acid; and a partially neutralized material thereof (acrylic acid units in a molecule are partially neutralized into salts); an IPN material between a crosslinked copolymer comprising at least a (meth)acrylamide and an N,N-dimethyl(meth)acrylamide, and crosslinked polymaleic acid; and a partially neutralized material thereof (acrylic acid units in a molecule are partially neutralized into salts); an IPN material between a crosslinked copolymer comprising at least a (meth)acrylamide and an N,N-dimethyl(meth)acrylamide, and crosslinked polyfumaric acid; and a partially neutralized material thereof (acrylic acid units in a molecule are partially neutralized into salts); and the like. However, the IPN materials are not limited to the above examples.

Specific examples of a block copolymer gel of polymers which form hydrogen bond to each other include a crosslinked copolymer comprising at least a (meth)acrylamide, an N,N-dimethyl(meth)acrylamide and a (meth)acrylic acid; a crosslinked copolymer comprising at least a (meth)acrylamide, an N,N-dimethyl(meth)acrylamide and a maleic acid; a crosslinked copolymer comprising at least a (meth)acrylamide, an N,N-dimethyl(meth)acrylamide and fumaric acid; and the like. However, the block copolymer gels are not limited to the above examples.

A volume change amount of a polymer gel caused by stimuli is preferably larger in terms of a light-modulation characteristic and a volume ratio of swelling state volume to shrinking state volume is 5 or larger, preferably 10 or larger and more preferably 15 or larger.

A temperature showing a volume change (a phase transition temperature) can be controlled by adding a water-soluble organic compound or an acidic compound into a swelling liquid. In addition thereto, various kinds of designs are enabled by changing a structure or a composition of a polymer gel. Here, a preferable range of transition temperatures is selected within a range of −30° C. to 300° C. and a phase transition temperature is especially preferably in the range of −10° C. to 200° C.

A physical form of a polymer gel and the like is not particularly limited, but considering a stimuli-response characteristic, it is especially preferable to use a polymer gel in a form of particles. The shape of a particle is not particularly limited, and a spherical particle, a ellipsoidal particle, a polyhydral particle, a porous particle, a fibrous particle, a star-like particle, a needle-like particle, a hollow particle or the like.

An average diameter of a polymer gel in dry state is preferably in the range of 0.01 μm to 5 mm and especially in the range of 0.01 μm to 1 mm. If an average particle diameter is less than 0.01 μm or less, an optical characteristic cannot be obtained, a polymer gel is easy to cause aggregation or the like and, handling becomes sometimes difficult when a person uses the polymer gel. On the other hand, if the average diameter is larger than 5 mm, unfavorably, a response speed sometimes becomes slower.

Particles of a polymer gel can be produced according to general particle-formation methods, examples of which are a method in which a polymer gel is made particles by physical pulverization, a method in which a polymer prior to crosslinking is made particles by chemical pulverization or the like, followed by crosslinking, and granular polymerization methods such as an emulsion polymerization method, a suspension polymerization method, a dispersion polymerization method and the like. Furthermore, polymer particles can also be produced in a method in which polymer prior to crosslinking is extruded through a nozzle into fibers and then fibers are crosslinked and pulverized, or in a method of pulverizing the fibers into particles, followed by crosslinking.

A polymer gel by itself shows a light-modulation property by changing a light scattering property according to a volume change, but it is preferable to add a light-modulation material inside of the polymer gel in order to exhibit a greater light-modulation characteristic and a greater color change.

As light-modulation materials to be added, a dye, a pigment, a light scattering material and the like can be cited. Furthermore, a light-modulation material is preferably fixed physically or chemically to a polymer gel.

Preferable specific examples of dyes include a black nigrosine type dye; an azo, a anthraquinone type dye, an indigo type dye, a phthalocyanine type dye, a carbonium dye, a quinoneimine dye, a methine dye, a quinoline dye, a nitro dye, a benzoquinone dye, a naphtoquinone dye, a naphthalimide dye, a berinone dye, which are color dyes for red, green, blue, cyan, magenta, yellow and the like, among which desirable is a dye having a high light absorption coefficient. Examples of dyes include C. I. direct yellow 1, 8, 11, 12, 24, 26, 27, 28, 33, 39, 44, 50, 58, 85, 86, 87, 88, 89, 98 and 157; C. I, acid yellow 1, 3, 7, 11, 17, 19, 23, 25, 29, 38, 44, 79, 127, 144 and 245; C. I. basic yellow 1, 2, 11 and 34; C. I. food yellow 4, C. I. reactive yellow 37; C. I. solvent yellow 6, 9, 17, 31, 35, 100, 102, 103 and 105: C. I. direct red 1, 2, 4, 9, 11, 13, 17, 20, 23, 24, 28, 31, 33, 37, 39, 44, 46, 62, 63, 75, 79, 80, 81, 83, 84, 89, 95, 99, 113, 197, 201, 218, 220, 224, 225, 226, 227, 228, 229, 230 and 231; C. I. acid red 1, 6, 8, 9, 13, 14, 18, 26, 27, 35, 37, 42, 52, 82, 85, 87, 89, 92, 97, 106, 111, 114, 115, 118, 134, 158, 186, 249, 254 and 289; C. I. basic red 1, 2, 9, 12, 14, 17, 18 and 37; C. I. food red 14, C. I. reactive red 23 and 180; C. I. solvent red 5, 16, 17, 18, 19, 22, 23, 143, 145, 146, 149, 150, 151, 157 and 158; C. I. direct blue 1, 2, 6, 15, 22, 25, 41, 71, 76, 78, 86, 87, 90, 98, 163, 165, 199 and 202; C. I. acid blue 1, 7, 9, 22, 23, 25, 29, 40, 41, 43, 45, 78, 80, 82, 92, 93, 127 and 249; C. I. basic blue 1, 3, 5, 7, 9, 22, 24, 25, 26, 28 and 29; C. I. food blue 2, C. I. solvent blue 22, 63, 78, 83 to 86, 191, 194, 195 and 104; C. I. direct black 2, 7, 19, 22, 24, 32, 38, 51, 56, 63, 71, 74, 75, 77, 108, 154, 168 and 171; C. I. acid black 1, 2, 7, 24, 26, 29, 31, 44, 48, 50, 52 and 94; C. I. basic black 2 and 8; C. I. food black 1 and 2; C. I. reactive black 31; C. I. food violet 2; C. I. solvent violet 31, 33 and 37; C. I. solvent green 24 and 25; C. I. solvent brown 3 and 9; and others. The dyes described above may be used either alone or in mixture of them in order to obtain a desired color.

Furthermore, in order to fix a dye to a polymer gel, there are preferably used a dye having a polymerizable group such as an unsaturated double bond group, a so-called reactive dye that can react with a polymer gel and the like. A preferable concentration of a dye included in a polymer gel is in the range of 3% by mass to 50% by mass and an especially preferably in the range of 5% by mass to 30% by mass. In such way, a dye concentration is desirably at least equal to or higher than a saturated absorption concentration in a dry state or a shrinking state of a polymer gel. Here, “higher than a saturated absorption concentration” represents a region of high dye concentrations where a relationship between a dye concentration and an optical density (or a light absorption amount) in a specified optical path length is greatly deviated from a linear relation.

Specific examples of the pigments and the light-scattering materials include bronze powder, titanium black, various kinds of carbon blacks (channel black, furnace black and the like), which are black pigments; metal oxides such as titanium oxide and silica, which are white pigments; light scattering materials such as calcium carbonate and metal powder; various pigments such as a phthalocyanine type cyan pigment, a banzidine type yellow pigment, a rhodamine type magenta pigment, which are color pigments; and in addition, other pigments and light scattering materials such as an anthraquinone type pigment, an azo type pigment, an azo metal complex, a phthalocyanine type pigment, a quinacridone type pigment, a perylene type pigment, an indigo type pigment, an isoindolinone type pigment, a quinacridone type pigment and an arylamide type pigment.

Representative examples of the yellow pigments that are preferably used in the invention include a condensation azo compound, an isoindolinone compound, an anthraquinone compound, an azo metal complex, a methine compound and an arylamide compound. More specifically, preferably used are C. I. pigment yellow 12, 13, 14, 15, 17, 62, 74, 83, 93, 94, 95, 109, 110, 111, 128, 129, 147, 168 and the like.

Examples of the magenta type pigments that can be used in the invention include a condensation azo compound, a diketo-pyrrolopyrrole compound, anthraquinone, a quinacridone compound, a lake pigment, a naphthol compound, a benzimidazolone compound, a thioindigo compound and a perylene compound. More specifically, especially preferable are C. I. pigment red 2, 3, 5, 6, 7, 23, 48:2, 48:3, 48:4, 57:1, 81:1, 144, 146, 166, 169, 177, 184, 185, 202, 206, 220, 221 and 254.

As cyan type pigments, a copper phthalocyanine compound and a derivative thereof, an anthraquinone compound, a basic dye lake compound and the like can be used. Specifically, C. I. pigment blue 1, 7, 15, 15:1, 15:2, 15:3, 15:4, 60, 62, 66 and the like can be preferably used as a pigment.

Particle diameters of a pigment and a light scattering material used in the invention are preferably in the range of from 0.001 μm to 1 μm and especially preferably in the range of 0.01 μm to 0.5 μm in terms of an average diameter of primary particles. This is because, if particle diameters are smaller than 0.01 μm, particles easily flow out of a polymer gel, while if they are larger than 0.5 μm, a color developing characteristic may be worsened.

As described above, a pigment and a light scattering material are required to be included in a polymer gel and prevented from flowing out of the polymer gel. Therefore, it is preferable that a crosslinking density of a polymer gel is optimized to physically confine a pigment and a light scattering material within a polymer network. It is also preferable to use a pigment or a light scattering material, which shows strong physical interactions such as electrical interaction and ionic interaction, or to use a pigment or a light scattering material, surfaces of which are chemically modified. As pigments and light scattering materials surfaces of which are chemically modified, a pigment and a light scattering material onto which an unsaturated group such as a vinyl group or a group that chemically bonds to a polymer gel such as an unpaired electron (radical) is introduced, and a pigment and a light scattering material to which a polymer gel is graft bonded, can be cited.

An amount of a pigment and a light scattering material contained in a polymer gel is, similar to the case of a dye, preferably at a concentration equal to or higher than a saturated absorption concentration (or equal to or higher than a saturated light scattering concentration) in a polymer gel in a state not containing a liquid.

Here, in order to make a concentration equal to or higher than a saturated absorption concentration (or equal to or higher than a saturated light scattering concentration), the concentration is generally in the range of 3% by mass to 95% by mass and preferably in the range of 5% by mass to 80% by mass. If a concentration of a pigment (or a light scattering material) is equal to or lower than 3% by mass, the concentration of a pigment (or a light scattering material) does not reach a value equal to or higher than a saturated absorption concentration (or equal to or higher than a saturated light scattering concentration), therefore a light-modulation characteristic accompanying a change in a volume of a polymer gel are not obtained. On the other hand, if the concentration of a pigment is equal to or higher than 95% by mass, a response speed and a volume change amount may be reduced.

A polymer gel including such a light-modulation material can be produced by a method in which a light-modulation material is uniformly dispersed and mixed into a polymer prior to crosslinking, followed by crosslinking or a method in which a light-modulation material is added into a polymer precursor monomer composition then, the monomer composition is polymerized. In the case where a pigment or a light scattering material is added at polymerization, it is preferable to use the pigment or the light scattering material having a polymerizable group or an unpaired electron (radical) to be chemically bonded to a polymer gel.

A light-modulation material is preferably dispersed in a polymer gel as uniformly as possible. Especially, in dispersing a light-modulation material into a polymer, it is desirable to uniformly disperse the light-modulation material into the polymer, employing a mechanical kneading method or an agitating method, or using a dispersant.

From such a viewpoint, as light-modulation materials, there can also preferably used light-modulation materials, having a polar group such as an acid group, an hydroxyl group, an amino group, a thiol group, a halogen atom, a nitro group, a carbonyl group or the like in a molecule, and having the characteristic that the light-modulation materials easily form an aggregate in the case where the light-modulation material concentration is high in a polymer gel. Examples of such light-modulation materials include phthalocyanine type pigments, azo type pigments and the like having an hydroxyl group, an amino group, a carboxyl group, or a sulfonic acid group. Furthermore, it is preferable to use various chemically-modified light-modulation materials, such as a light-modulation material having an group that can perform an addition reaction or a polymerizable group to form a covalent bond to a polymer gel, and a light-modulation material having a group that shows interaction with a polymer gel such as an ionic bond.

(Swelling Liquid)

As swelling liquids, there are preferably exemplified: water, a water-soluble organic compound, and a mixture obtained by further adding an acid compound to a mixture thereof. Especially, by adding a water-soluble organic compound into a swelling liquid, hydrolysis of an amide group included in a polymer gel is suppressed, thereby preventing degradation in characteristics at a high temperature more efficiently. Furthermore, in order to control a condition that suppresses the hydrolysis, it is preferable to adjust a pH value of a swelling liquid. And, it is preferable to add an acidic compound into swelling liquid as a pH adjusting agent. Hereinafter, detailed description will be given of a water-soluble organic compound and an acidic compound.

As water-soluble organic compounds, there are exemplified: an alcohol, a keton, an ether, an ester, an amide, a polymer including a functional group described above in a repeating unit and the like. More specifically, alcohols such as methyl alcohol, ethyl alcohol, propyl alcohol, butyl alcohol, ethylene glycol, propylene glycol, ethylene glycol monoethyl ether and the like; ketones, such as acetone, methyl ethyl ketone and the like; ethers such as THF, 1,4-dioxane, diethyl ether, ethylene glycol diethyl ether and the like; esters such as ethyl acetate and the like; and dimethylformamide, dimethylacetamide, dimethylsulfoxide, acetonitrile, urea and the like can be cited. Furthermore, there are also preferably used: polyvinyl alcohol, a poly(meth)acrylamide and derivatives thereof; polyvinylpyrrolidone, polyethylene oxide and coplymers including such polymers. Among them, preferable are alcohols such as methyl alcohol, ethyl alcohol, propyl alcohol, butyl alcohol, ethylene glycol, propylene glycol, ethylene glycol monoethyl ether and the like; dimethylsulfoxide, dimethylformamide and the like, and especially preferable are alcohols.

An amount of a water-soluble organic compound to be added is approximately in the range of 5 to 50% by mass and preferably in the range of 10 to 40% by mass.

On the other hand, as acidic compounds, an inorganic acid, an organic acid, acidic polymer and the like can be cited. Specifically, inorganic acids such as hydrochloric acid, sulfuric acid, phosphoric acid, nitric acid and the like; organic acids such as acetic acid, propionic acid, butylic acid, valeric acid, benzoic acid, phenylacetic acid, acrylic acid, maleic acid, fumaric acid, dimethylsulfuric acid, diethylsulfuric acid, p-toluenesulfonic acid and the like; and acidic polymers such as polyvinylsulfonic acid, polyphosphoric acid, a poly(meth)acrylic acid, poly-L-glutamic acid, polymaleic acid, polyfumaric acid, and copolymers including at least one acidic polymer described above can be cited. Among them, further preferable are inorganic acids such as hydrochloric acid and phosphoric acid; organic acids such as acetic acid and the like; and acidic polymers exemplified above. More preferable are a poly(meth)acrylic acid including a carboxyl group in a repeating unit in a molecule; and copolymers including poly-L-glutamic acid, polymaleic acid, polyfumaric acid, or a poly(meth)acrylic acid. A molecular weight of an acidic polymer is preferably in the range of from 500 to 200,0000 and more preferably in the range of from 1,000 to 100,0000 in terms of weight average molecular weight.

An amount of an acidic compound to be added is approximately in the range of from 0.001 to 50% by mass and more preferably, in the range of 0.01% by mass to 30% by mass.

A pH value of a swelling liquid is preferably in the range of 2 to 7, and more preferably in the range of from 2 to 5. A pH value in the range prevents degradation in characteristics at a high temperature.

There may be added various agents in a swelling liquid such as a surfactant adsorbed to and desorbed from a polymer gel, a redox agent such as a viologen derivative for accelerating a change in pH value of a solution, an acid, an alkali, a base and a dispersion stabilizer, an anticeptic agent, an antibacterial agent, stabilizers such as an antioxidant, an ultraviolet ray absorber and the like. Further, colorants such as various kinds of pigments, a white pigment, a dye and the like can be added.

A mixing ratio of a polymer gel to a swelling liquid is preferably in the range of from 1/2000 to 1/1 (polymer gel/swelling liquid) by mass. If the mass ratio is less than 1/2000, a degradation in a physical property such as a mechanical strength of a composition may take place, while if the ratio is higher than 1/1, reduction in a response speed of a volume change to be caused by stimuli-response may take place.

(Usage Modes of Polymer Gel Composition)

As usage modes of a polymer gel of the invention, a mode (an optical device) in which a polymer gel composition is sandwiched (or sealed) between a pair of substrates; a mode (resin composition) in which a polymer gel composition is dispersed in a resin; a mode (an optical film) in which a resin with a polymer gel of the invention dispersed therein is formed on a film; and the like can be cited. In the case where a polymer gel composition is dispersed in a resin, a resin composition can be prepared by mixing a polymer gel composition into a resin or a resin precursor with which the composition is incompatible, drying the resin, and polymerizing or curing the resin. Furthermore, a polymer gel composition may be microencapsulated with a polymer film, then dispersed into a resin. In such a way, it is possible to expand usage modes of the polymer gel composition.

Further detailed description of usage modes of a polymer gel composition of the invention will be given below with reference to the drawings.

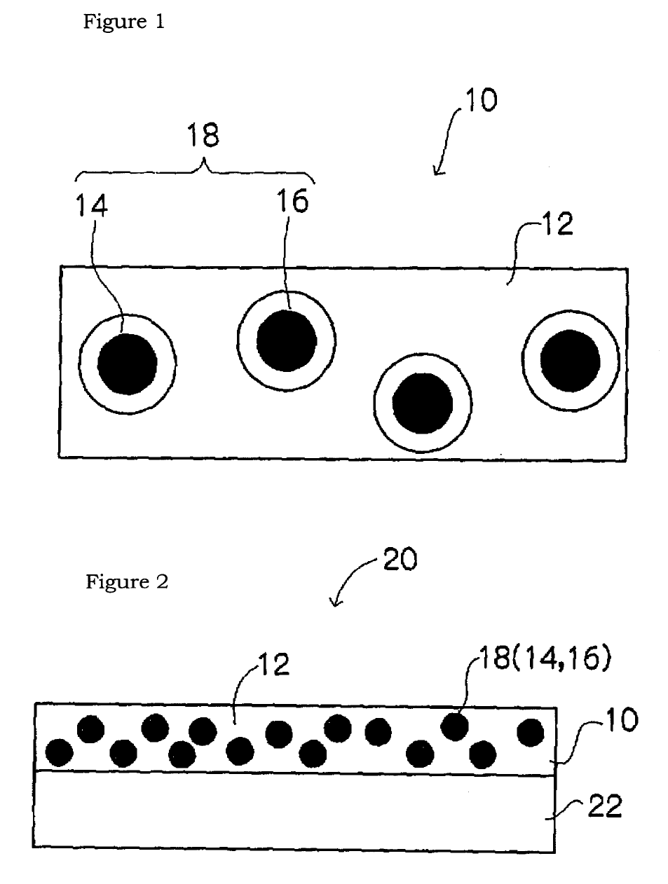

A resin composition 10 shown in FIG. 1 is one embodiment in which a polymer gel composition 18 including a polymer gel 14 and a swelling liquid 16 is dispersed in a resin 12. It is also possible to micro-encapsulate a polymer gel composition 18 including a polymer gel 14, and a swelling liquid 16 including a water-soluble organic compound and an acidic compound and disperse the polymer gel composition 18 in microcapsules in a resin.

Here, a composition ratio of a resin 12 to a polymer gel composition 18 (for example, a composition composed of a polymer gel 14+a swelling liquid 16 including a water-soluble organic compound and an acidic compound) is preferably in the range of 1/50 to 50/1 [resin 12/(polymer gel 14+swelling liquid 16 including a water-soluble organic compound and an acidic compound), or resin 12 (a matrix material)/(capsules containing a polymer gel 14+a swelling liquid 16 including a water soluble organic compound and an acidic compound)] by mass.

Here, a polymer gel composition can be micro-encapsulated by a so-called coacervation method using insolubilization of a polymer material; a so-called interfacial polymerization micro-encapsulation method in which polymerization is performed at an interface of dispersed particles to form a capsule film, an in-situ micro-encapsulating polymerization method, an in-liquid drying method and an in-liquid cured coating micro-encapsulation method; and a spray drying micro-encapsulation method in which liquid droplets are sprayed in a gas to form a capsule film on surfaces of the droplets; and the like. Details of the techniques exemplified above are described in, for example, a book entitled “Shinban Maikurokapuseru, Sonoseihou Seishitsu Ouyou” (New version Microcapsule, its Production Method, Properties and Applications), authored by Tamotsu KONDO and published by Sankyo Publication. A thickness of a polymer film constituting a capsule is preferably in the range of 1 nm to 20 μm and more preferably in the range of 1 nm to 10 μm. A size of a capsule is preferably in the range of 1 μm to 5 mm and more preferably in the range of 10 μm to 2 mm in terms of average diameter.

A mixing ratio of a capsule material to a polymer gel composition (a mixture of a polymer gel and a swelling liquid) is preferably in the range of 1/200 to 5/1 [capsule material/(polymer gel and liquid)] by mass. Encapsulated resin composition can be used in various applications as it is or by dispersed into another resin.

As resins 12 (a matrix material) described above, a heat-curable resin, an ultraviolet ray-curable resin, an electron curable resin, a polyester, a polyimide, acrylic resins such as polymethylmethacrylate, polystyrene and a derivative thereof, polyethylene, polypropylene, a polyamide, polyvinyl chloride, polyvinylidene chloride, polycarbonate, polyethersulfone, a cellulose derivative, a fluorine type resin, a silicone type resin, an epoxy type resin, a polyacetal type resin and the like can be cited. The resin is preferably optically transparent. Furthermore, the resin may be in a state of mixtures with various kinds of solvents or in a state of a gel. The resin is particularly preferably a resin having high transmittance to light. In order to reduce light scattering at an interface between a liquid including a polymer gel and the resin described above to raise transparency, a difference between refractive indexes of a liquid including a polymer gel and the resin, respectively, is preferably adjusted to be 0.2 or less. The difference in the refractive indexes is more preferably 0.1 or less and especially preferably 0.01 or less.

Such a resin composition 10 can be used in various structures such as a film, a fiber and others. Especially, in the case where it is used in a film, the composition is formed on various film substrates or sandwiched between plural film substrates to have a specified thickness, thereby enabling a light-modulation film excellent in stability and durability to be obtained.

An optical film 20 shown in FIG. 2 is a structural example provided with a layered resin composition 10 on a film substrate 22 in which composition, a polymer gel composition 18, composed of a polymer gel 14 (not shown) and a swelling liquid 16 (not shown), is dispersed. In addition thereto, there may be formed a structure in which the composition is sandwiched between two film substrates 22, and a structure in which another structural layer such as a protective layer, a layer that prevents an evaporation of the swelling liquid 16, or the like is provided on the film.

As film substrates 22, polyester, polyimide, polymethylmethacrylate, polystyrene, polypropylene, polyethylene, polyamide, polyvinyl chloride, polyvinylidene chloride, polycarbonate, polyethersulfone, a cellulose derivative, a fluorine type resin, a silicone type resin, an epoxy type resin, a polyacetal type resin, a metal film and the like can be used.

In the optical film 20 shown in FIG. 2, as to specific thicknesses preferable for respective layers, a thickness of the film substrate 22 may be in the range of 10 μm to 10 mm and a thickness of the resin composition 10 layer (a light-modulation resin composition layer) may be in the range of 5 μm to 10 mm. Furthermore, in the optical film 20 shown in FIG. 20, it is possible that a pair of substrates 32 described later is used instead of the film substrate 22 and the resin composition 10 is sandwiched between the pair of substrates 32, thereby forming an optical device.

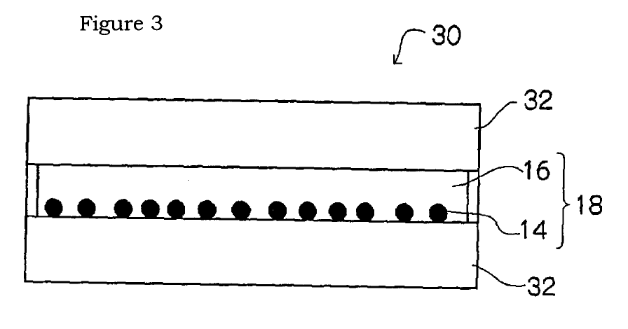

An optical device 30 shown in FIG. 3 is an structure example in which a polymer gel composition 18 composed of a polymer gel 14 and a swelling liquid 16 is sealed between two substrates 32. In this structure, plural kinds of polymer gels 14 in the polymer gel composition 18 are preferably fixed onto a substrate 32 (or a stimulating means described later). Fixing of the polymer gel 18 (gel particles) can be realized by using various kinds of di-functional compounds or adhesives, or by physical means. For example, a surface of a substrate or a stimulating layer is treated with a reactive silane coupling agent in advance to thereby introduce a functional group thereto, thereby forming a covalent bond or the like through a reaction of a functional group of a gel particle with the introduced functional group. As other fixing measures, it is possible to employ a method in which a gel particle is fixed by a chemical bond (an ionic bond and a hydrogen bond) with various kinds of polyfunctional compound or an adhesive and; a method in which a gel particle is physically fixed by processing the surface of a substrate three-dimensionally or by utilizing adhesion property of a gel particle. Here, if a gel particle is adhered to a substrate or the like to an excessive extent in fixing of the gel particles, a response characteristic are sometimes lowered; therefore, means in which a surface of a substrate or the like is three-dimensionally processed in order to keep a space thereon to cause gel particles to bond to protrusions on the surface of the substrate, or means in which gel particles are bonded to a substrate via a long chain compound (a spacer) interposed between them so as to provide a space, are preferably employed.

As substrates 32, a polymer film and a polymer plate substrate made of polyester, polyimide, polymethylmethacrylate, polystyrene, polypropylene, polyethylene, polyamide, polyvinyl chloride, polyvinylidene chloride, polycarbonate, polyethersulfone, a cellulose derivative, a silicon type resin, an epoxy type resin, a polyacetal type resin or the like; and an inorganic substrate such as a glass substrate, a metal substrate, a ceramic substrate or the like, can be used.

Note that at least one of substrates 32 is required to be optically transparent. Furthermore, in the case of a transparent optical device, all substrates are preferably transparent.

While substrates 32 of various thicknesses and sizes can be used in accordance with a desired display device without specific limitation placed thereon, the thickness is preferably in the range of 10 μm to 20 mm.

While the resin composition shown in FIG. 1, the optical film shown in FIG. 2 and the optical device shown in FIG. 3 can perform light-modulation and display due to a change in natural energy such as atmospheric temperature and a solar ray amount, it is also possible to intentionally modulate light with them by providing stimulating means. In this case, the stimulating means practically provide a heat to a polymer gel, examples of which include various kinds of heat feeding means such as feeding light, feeding electromagnetic wave, and feeding magnetic field, in addition to a current heating registor. Among them, especially preferable is current heating registor. Specifically, a metal layer such as Ni—Cr alloy; a metal oxide layer such as tantalum boride, tantalum nitride, tantalum oxide, and ITO; and a heating registor layer such as a carbon layer can be preferably used, and they are allowed to generate heat by providing wires in the layers and feeding current to the wires. Furthermore, in the case of feeding light, heat feeding means can be realized by using a layer that includes a light emitting device such as a laser, an LED, an EL and the like. And in the case of feeding magnetic field or electromagnetic wave, heat feeding means can be realized by providing an electromagnetic coil or electrodes.

It is preferable that the thermally stimulating means are patterned into segments to perform light-modulation at any portion. Furthermore, it is also preferable to arrange (fix) the polymer gels with specific characteristics in accordance with the pattern.

Furthermore, various structural layers may be formed in a resin composition, an optical film and an optical. For example, a protective layer for the purpose of protection, a contamination preventing layer, an ultraviolet absorbing layer, an antistatic layer and the like can be formed.

Description will be given of a typical fabrication method for the optical device shown in FIG. 3 as an example. Note that the description is given without using signs indicating constituent members.

Two substrates are prepared and a polymer gel (gel particles) are fixed on a surface of at least one of the substrates according to the method described above. Then, the substrates are stuck to each other with a specific space interposed between them to form a cell. The space between the substrates at this time is generally in the range of 5 μm to 10 mm. The space between the two substrates is set preferably by dispersing spacer particles with various sizes, using a film spacer, or utilizing a three-dimensional structure formed on the substrate or the like. Here, in the case where two substrates are stuck to each other, it is preferable to seal the periphery, except for a specific open portion, with a sealing resin, an adhesive, an ultraviolet ray-curable resin or a heat-curable resin. A liquid is injected through the open portion left locally unsealed after the sealing, according to a low-pressure injection method or the like, followed by sealing of the open portion with a similar resin to obtain the optical device.

### EXAMPLES

Further specific description will be given of the present invention by way of examples shown below. However, the invention is by no means limited by the examples.

### Example 1

(Production of Polymer Gel Particle A)

Particles of a thermo-sensitive type (high temperature swelling type) polymer gel containing a colorant were produced according to a process described below. An aqueous solution was prepared by adding 0.575 g of distilled water and 3.425 g of an aqueous dispersion containing a blue pigment as a colorant (micro-encapsulated blue pigment manufactured by DAINIPPON INK & CHEMICALS INC.) at a concentration of 13.5% by weight to 0.8 g of acrylamide, 0.2 g of N.N-dimethylacrylamide and 1.0 mg of methylenebisacrylamide as a crosslinker, and the solution was stirred and mixed. A solution prepared by dissolving 3.9 g of a sorbitol type surfactant (manufactured by Nikko Chemicals Co., Ltd. with a trade name of SO-15R) into 300 ml of cyclohexane was put into a reaction vessel which had been purged with nitrogen, the previously prepared aqueous solution was added into the vessel, and the mixture was stirred with a rotary agitating blade for 30 min at 1200 rpm to make a suspension.

The suspension was put into a flask and after oxygen was removed by purging with nitrogen, an aqueous solution obtained by dissolving 0.004 g of ammonium persulfate, which was a polymerization initiator, into 0.5 ml of wafer was added into the suspension. Then, the suspension was heated and kept at 60° C. for 30 min for polymerization. After the polymerization was completed, thus obtained polymer was purified by being washed with a large amount of acetone, then, dried to obtain an acrylamide gel containing a colorant. Then, 5.5 g of distilled water, 1.5 g of acrylic acid, and 0.0015 g of methylenebisacrylamide as a crosslinker were mixed. After an atmosphere was purged with nitrogen, a solution obtained by dissolving 0.006 g of ammonium persulfate into 0.5 g of water was further added into the mixed solution. To the mixed solution, 0.5 g of particles of said acrylamide gel was added, then the mixed solution was heated and kept at 70° C. to perform polymerization for 3 hr to prepare an IPN polymer gel. The IPN polymer gel particle was put into a large amount of distilled water, and the procedure was repeated for purifying the IPN polymer gel particle, in which procedure the IPN polymer gel particle was heated and cooled to swell and shrink and filtrated. A wall diameter of the obtained IPN gel particle in a dry state was about 15 μm. The IPN gel particles were swollen by being added to a large amount of pure water. An amount of absorbed water of the swelling gel particle at equilibrium at temperature of 10° C. was about 3 g/g (water/gel). It was found that the IPN gel particles further swelled to show about 60 g/g (water/gel) of water absorption when heated to 50° C. The phase transition temperature was in the range of 40 to 50° C. That is, the IPN gel particle swelled at a temperature higher than the phase transition temperature, while they shrank at a temperature lower than the phase transition temperature. This change was reversible and a diameter of a particle changed by a factor of about 2.7 (that is a volume changed by a factor of about 20) between swelling state and shrinking state.

(Production of Polymer Gel Composition and Optical Device J1)

An optical device using a polymer gel composition was produced by the following process.

Two glass substrates (each having a size of 50 mm×50 mm and a thickness of 3 mm) were prepared. γ-aminopropyltriethoxysilane was coated on a surface of one glass substrate and dried to form an adhesive layer for gel particles. On the other hand, in order to fix the gel particles, polymer gel particles A was immersed in an aqueous solution of 0.2% by weight polyacrylic acid (weight average molecular weight of 25000) to obtain a dispersion containing 1% by mass of solids.

One of the glass substrates was placed in a plastic vessel with a glass substrate surface having the adhesive layer thereon facing upward. The dispersion was poured onto the glass substrate and the plastic vessel was left at 60° C. for 15 hr, to fix the gel particles on the surface of the glass substrate. The glass substrate was washed with an aqueous solution 0.2% by weight polyacrylic acid (weight average molecular weight of 25000) to remove the stacked gel particles, thus the glass substrate on which the gel particles were fixed almost in one layer was obtained.

When the surface of the glass substrate was observed with a microscope, an area ratio of the portion on which the gel particles were fixed was about 70% at 60° C. An area ratio was defined as a proportion of an area of projection of the particles on the substrate to the area of the surface of the glass substrate.

A resin spacer having a particle size of 100 μm was sprinkled on a surface of the other glass substrate. Then, an ultraviolet ray-curable resin was coated along the outer periphery except for a local opening. The surface of the substrate on which the gel particles were fixed was closely superposed on the surface of the other surface, and an ultraviolet ray was irradiated onto the substrates to adhere them to each other.

Then, a mixed solution (a swelling liquid with a pH value of 3) of 3 g of methanol and 7 g of an aqueous solution containing 7% by weight of polyacrylic acid (weight average molecular weight of 25000) which was neutralized with NaOH by 3 mol %, was injected into of the cell, then, the opening was sealed to produce an optical device J1.

(Evaluation of Function)

The obtained optical device J1 was transparent at 10° C. and blue when heated to 60° C. When being cooled again to 10° C., the optical device returned to the initial transparent state, which shows that the color change was reversible. The optical device was immersed in an aqueous bath at 80° C. for 30 days. Then, the optical device was heated and cooled to show the reversible color change similar to that before heated for 30 days.

### Example 2

(Production of Polymer Gel Particle B and Optical Device J2)

Polymer gel particles B were prepared in a similar manner to Example 1 with the exception that N,N-dimethylacrylamide as a copolymerization monomer was replaced with methyl acrylate. A wall diameter of the obtained particle in a dry state was about 15 μm. The IPN gel particles were swollen by being added into a large amount of pure water. An amount of the absorbed water of the swelling IPN gel particles at equilibrium at temperature of 10° C. was about 2 g/g (water/gel). It was found that the IPN gel particles further swelled to show the water absorption of 20 g/g (water/gel) when heated to 50° C. The phase transition temperature was in the range of 40° C. to 50° C. That is, the IPN gel particles swelled at a temperature higher than the phase transition temperature, while the IPN gel particles shrank at a temperature lower than the phase transition temperature. This change was reversible and a diameter of the particle changed by a factor of about 2.2 between swelling state and shrinking state, that is a volume of the particle changed by a factor of about 10.

An optical device J2 was produced in a similar manner to Example 1 with the exception that the polymer gel particles B were used.

(Evaluation of Function)

The obtained optical device J2 was transparent at 10° C. and blue when heated to 60° C. When cooled again to 10° C., the optical device returned to the initial transparent state, which shows that the color change was reversible. The optical device was immersed in an aqueous bath at 80° C. for 30 days. Then, the optical device was heated and cooled to show the reversible color change similar to that before heated for 30 days.

### Example 3

(Production of Polymer Gel Particle C and Optical Device J3)

Polymer gel particles C were prepared in a similar manner to Example 1 with the exception that N,N-dimethylacrylamide as a copolymerization monomer was replaced with ethyl acrylamide. A wall diameter of the obtained particle in a dry state was about 15 μm. The particles of the IPN gel were swollen by being added to a large amount of pure water. An amount of absorbed water of the swelling gel at equilibrium at temperature of 10° C. was about 2 g/g (water/gel). It was found that the IPN gel particles swelled further to show the water absorption of 30 g/g (water/gel) when heated to 50° C. The phase transition temperature was in the range of 40 to 50° C. That is, the gel swelled at a temperature higher than the phase transition temperature, while the gel shrank at a temperature lower than the phase transition temperature. This change was reversible and a diameter of the particle changed by a factor of about 2.5 between swelling state and shrinking state, that is a volume of the particle changed by a factor of about 16.

An optical device J3 was produced in a similar manner to Example 1 with the exception that the polymer gel particles C were used.

(Evaluation of Function)

The obtained optical device J3 was transparent at 10° C. and blue when heated to 60° C. When cooled again to 10° C., the optical device returned to the initial transparent state, which shows that the color change was reversible. The optical device was immersed in an aqueous bath at 80° C. for 30 days. Then, the optical device was heated and cooled to show the reversible color change similar to that before heated for 30 days.

### Example 4

(Production of Resin Composition E and Optical Device J4)

0.5 g of glycidyl methacrylate was added to 20 g of an aqueous solution of 20% by weight of polyacrylic acid (manufactured by Wako Pure Chemical Industries, Ltd. with a weight average molecular weight of 250,000) to perform a reaction at room temperature for 24 hr under agitation. To the solution, 0.8 g of a photoinitiator (manufactured by Chiba Specialty Chemical Inc., with a trade name of Ilgacure 2959), 40 g of pure water and 24 g of methanol were added to prepare a resin composition E. A pH value of the resin composition was about 2.8 [a liquid present in the resin after the resin composition was cured (an aqueous solution prepared by adding 24 g of methanol to 56 g of water) functions as a swelling liquid]. A layer of the resin composition E having a thickness of 100 μm was sandwiched between glass substrates and irradiated with an ultraviolet ray (using a high pressure mercury lamp at an output of 160 W/cm for 150 sec with an irradiation distance of 40 cm). Thus, the entire resin composition became a gel to obtain a cured resin with a self-supporting property.

Then, prepared was an aqueous dispersion containing the polymer gel particles A prepared in Example 1 at a certain concentration (at a solids concentration of the gel of 2.5%). 10 ml of said aqueous dispersion containing particles A was added to 10 g of the resin composition E, and the mixture was treated with a wave rotor for 3 hr to uniformly disperse the polymer gel particles A into the solution (dispersion A).

The dispersion A was coated on a PET substrate using a blade coater to have a thickness of 150 μm and laminated with another PET film. The laminate was irradiated with an ultraviolet ray (using a high pressure mercury lamp at an output of 160 W/cm for 120 sec with an irradiation distance of 20 cm), and the dispersion was cured. Then, the peripheral portion was sealed with a thermoplastic, photosensitive acrylic type adhesive (manufactured by Nippon Kayaku Co., Ltd. with a trade name of KAYARAD R381 1) to produce an optical device J4.

(Evaluation of Function)

The obtained optical device J4 was transparent at 10° C. and blue when heated to 60° C. When cooled again to 10° C., the optical device returned to the initial transparent state, which shows that the color change was reversible. The optical device was immersed in an aqueous bath at 80° C. for 30 days. Then, the optical device was heated and cooled to show the reversible color change similar to that before heated for 30 days.

### Example 5

An optical device J5 was produced in a similar manner to Example 1 with the exception that a mixed solution (with a pH value of 3) of 3 g of ethanol and 7 g of a 0.2% PAAc (having a weight average molecular weight of 250,000) aqueous solution, was used as a swelling liquid for a polymer gel.

(Evaluation of Function)

The obtained optical device J5 was transparent at 10° C. and blue when heated to 60° C.. When cooled again to 10° C., the optical device returned to the initial transparent state, which shows that the color change was reversible. The optical device was immersed in an aqueous bath at 80° C. for 30 days. Then, the optical device was heated and cooled to show the reversible color change similar to that before heated for 30 days.

### Example 6

An optical device J6 was produced in a similar manner to Example 1 with the exception that a mixed solution (with a pH value of 3) of 3 g of methanol and a 0.2% PAAc (with a weight average molecular weight of 250,000) aqueous solution, was used as a swelling liquid for a polymer gel.

(Evaluation of Function)

The obtained optical device J6 was transparent at 10° C. and blue when heated to 60° C.. When cooled again to 10° C., the optical device returned to the initial transparent state, which shows that the color change was reversible. The optical device was immersed in an aqueous bath at 80° C. for 30 days. Then, the optical device was heated and cooled to show the reversible color change similar to that before heated for 30 days.

### Example 7

An optical device J7 was produced in a similar manner to Example 1 with the exception that a 5% by weight polyacrylic acid (having a weight average molecular weight of 25,000, neutralized with NaOH by 3 mol %) aqueous solution (with a pH value of 3) was used as a swelling liquid for a polymer gel.

(Evaluation of Function)

The obtained optical device J7 was transparent at 10° C. and blue when heated to 60° C. When cooled again to 10° C., the optical device returned to the initial transparent state, which shows that the color change was reversible. The optical device was immersed in an aqueous bath at 80° C. for 30 days. Then, the optical device was heated and cooled to show a reversible color change. However, a degree of the color change was somewhat smaller when visually observed than the color change before heated for 30 days.

### Comparative Example 1

(Production of Glass Cell Including Non-Copolymer Type Colored Polymer Gel Particles)

Polymer gel particles and an optical device J7 were obtained in a similar manner to Example 1 with the exception that 1.0 g of acrylamide was used without adding N,N-dimethylacrylamide as a copolymerization monomer.

(Evaluation of Function) The obtained optical device J7 was transparent at 10° C. and blue when heated to 60° C. When cooled again to 10° C., the optical device returned to the initial transparent state, which shows that the color change was reversible. The optical device was immersed in an aqueous bath at 80° C. for 30 days. Then, the optical device was heated and cooled. However a color of the optical device was constantly blue with no change in color regardless of the heating and the cooling.

According to the invention, as described above, there can be provided a polymer gel composition, sustaining a light-modulation performance over a long term without reduction and excellent in durability even at a high temperature and an optical device, a resin composition and an optical film, using the polymer gel composition.

What is claimed is:

1. A polymer gel composition comprising:
a swelling liquid; and
a polymer gel which has the characteristic of absorbing and releasing the swelling liquid due to a change in temperature so as to reversibly change a volume of the polymer gel, and includes a crosslinked polymer having at least a hydrogen bonding group,
wherein the crosslinked polymer included in the polymer gel is a copolymer of at least two monomer components including;
(meth)acrylamide or a mono-substituted derivative thereof as a monomer component (A); and
a monomer different from the monomer component (A) as a monomer component (B).

2. A polymer gel composition according to claim 1, wherein the monomer component (A) is (meth)acrylamide, and the monomer component (B) is selected from the group consisting of a mono-substituted (meth)acrylamide, a di-substituted (meth)acrylamide, a (meth)acrylate derivative and a vinyl type monomer.

3. A polymer gel composition according to claim 1, wherein the monomer component (A) is (meth)acrylamide, and the monomer component (B) is selected from the group consisting of a mono-substituted (meth)acrylamide, a di-substituted (meth)acrylamide, and a (meth)acrylate derivative.

4. A polymer gel composition according to claim 1, wherein the monomer component (A) is a mono-substituted derivative of (meth)acrylamide, and the monomer component (B) is selected from the group consisting of (meth)acrylamide, a di-substituted (meth)acrylamide, a (meth)acrylate derivative, and a vinyl type monomer.

5. A polymer gel composition according to claim 1, wherein the monomer component (A) is a mono-substituted derivative of (meth)acrylamide, and the monomer component (B) is selected from a group consisting of (meth)acrylamide, a di-substituted (meth)acrylamide, and a (meth)acrylate derivative.

6. A polymer gel composition according to claim 1, wherein a pH value of the swelling liquid is from 2 to 7.

7. A polymer gel composition according to claim 1, wherein the swelling liquid includes at least a water-soluble organic compound.

8. A polymer gel composition according to claim 7, wherein the water-soluble organic compound is an alcohol.

9. A polymer gel composition according to claim 1, wherein the swelling liquid includes at least an acidic compound.

10. A polymer gel composition according to claim 9, wherein the acidic compound is a polymer having a carboxyl group.

11. A polymer gel composition according to claim 1, wherein the polymer gel is an IPN material including at least polymers which form hydrogen bonds with each other.

12. A polymer gel composition according to claim 11, wherein the IPN material comprises the crosslinked polymer having at least a hydrogen bonding group, and a crosslinked polymer having at least a carboxylic group or a partially neutralized crosslinked polymer of the crosslinked polymer having at least a carboxylic group.

13. A polymer gel composition according to claim 1, wherein the swelling liquid includes at least an alcohol, and the polymer gel is an IPN material comprising the crosslinked polymer having at least a hydrogen bonding group, and a crosslinked polymer having at least a carboxylic group or a partially neutralized crosslinked polymer of the crosslinked polymer having at least a carboxylic group.

14. A polymer gel composition according to claim 1, wherein the swelling liquid includes a polymer having at least a carboxyl group, and the polymer gel is an IPN material comprising the crosslinked polymer having at least a hydrogen bonding group, and a crosslinked polymer having at least a carboxylic group or a partially neutralized crosslinked polymer of the crosslinked polymer having at least a carboxylic group.

15. A polymer gel composition according to claim 1, wherein the polymer gel contains a light-modulation material therein.

16. An optical device comprising:
a polymer gel composition comprising
a swelling liquid, and
a polymer gel which has the characteristic of absorbing and releasing the swelling liquid due to a change in temperature so as to reversibly change a volume of the polymer gel, and includes a crosslinked polymer having at least a hydrogen bonding group; and
a substrate sandwiching the polymer gel composition,
wherein the crosslinked polymer included in the polymer gel is a copolymer of at least two monomer components including
(meth)acrylamide or a mono-substituted derivative thereof as a monomer component (A); and
a monomer different from the monomer component (A) as a monomer component (B).

17. A resin composition comprising:
a polymer gel composition comprising
a swelling liquid, and
a polymer gel which has the characteristic of absorbing and releasing the swelling liquid due to a change in temperature so as to reversibly change a volume of the polymer gel, and includes a crosslinked polymer having at least a hydrogen bonding group; and
a resin in which the polymer gel composition is dispersed,
wherein the crosslinked polymer included in the polymer gel is a copolymer of at least two monomer components including
(meth)acrylamide or a mono-substituted derivative thereof as a monomer component (A); and
a monomer different from the monomer component (A) as a monomer component (B).

18. An optical film comprising:
a polymer gel composition comprising
a swelling liquid, and
a polymer gel which has the characteristic of absorbing and releasing the swelling liquid due to a change in temperature so as to reversibly change a volume of the polymer gel, and includes a crosslinked polymer having at least a hydrogen bonding group; and
a film on which the polymer gel composition is provided,
wherein the crosslinked polymer included in the polymer gel is a copolymer of at least two monomer components including
(meth)acrylamide or a mono-substituted derivative thereof as a monomer component (A); and
a monomer different from the monomer component (A) as a monomer component (B).
